(19) United States
(12) Patent Application Publication
(10) Pub. No.: US 2005/0228503 A1
(43) Pub. Date: Oct. 13, 2005

(54) Device for stimulating bone growth, especially for the osteosynthesis of bone fragments and/or for fixing bone fractures

(76) Inventor: Ferdinand Gundolf, Kufstein (AT)

Correspondence Address: KNOBBE MARTENS OLSON & BEAR LLP, 2040 MAIN STREET, FOURTEENTH FLOOR, IRVINE, CA, 92614, US

(21) Appl. No.: 10/511,116

(22) PCT Filed: Apr. 9, 2003
(86) PCT No.: PCT/EP03/03690

§ 371 Date: Apr. 25, 2005

(30) Foreign Application Priority Data
Apr. 11, 2002 (DE) 10215996.3

(51) Int. Cl. A61F 2/34; A61F 2/28; A61B 17/84

(52) U.S. Cl. 623/22.21; 623/23.49; 606/72; 606/76; 623/22.35; 607/51

(57) ABSTRACT

Apparatus for promoting bone growth, especially for osteosynthesis of bone fragments and/or fixation of bone fractures is described. This apparatus comprises at least one piezoelectric element, which is associated with an implant or like bone fixation means and which, under the action of forces, generates electrical pulses which serve as a stimulant for bone growth. The at least one piezoelectric element is an integral component of the implant.

The invention relates to an apparatus for promoting bone growth, especially for osteosynthesis of bone fragments and/or fixation of bone fractures, according to the preamble of claim 1.

The concern in the present case is to promote bone growth, especially in the field of bone fractures, but also for the purpose of reducing osteoporosis, which is increasingly becoming an economic requirement. In austria alone, for example, 150 million euros per year are having to be spent on caring for femoral neck fractures, with the secondary costs not having been included in that figure. On average, every third woman from 60 to 70 years of age suffers from osteoporosis and, among the over-80's, it is even ⅔ of all women that are affected. Osteoporosis-related fractures result in immobility and a need to be cared for, pain and loss of quality of life. The mortality rate during the rehabilitation phase is high. The medical costs for treating osteoporosis-related fractures to be met annually in the usa and europe are currently about 25 billion euros. That figure does not include the indirect secondary costs such as the costs for rehabilitation and care, for sick-leave, loss of work and long-term institutional care.

There is accordingly a huge need for a remedy and for reducing the afore-mentioned costs.

From de 4 102 462 a1, which was originated by the inventor, there is known a purely mechanical apparatus for promoting bone growth. The usually elongate stabilising element described therein for the osteosynthesis of bone fragments has, despite its thin-walled construction, a high degree of rigidity, that being brought about by a cross-section of arcuate, wavy, meandering, zigzag or like shape. It has been shown in practice that the said stabilising element is also well-tolerated and, in addition, simple to implement. The said stabilising element has been found to be especially suitable for mechanical support and/or assistance in the healing of complex bone fractures. As a result of the fact that the known stabilising element has only linear contact with the associated bone and is made from biologically tolerable material such as, for example, titanium or titanium alloy, which is preferably roughened on the surface, bone growth is promoted in positive manner.

Alternative investigations have shown that bone growth can be further promoted by electrical stimulation. For that purpose, two different methods have been used hitherto:

The electrical stimulation can be carried out, on the one hand, directly by means of conductive coupling by way of supply wires or, on the other hand, inductively by way of an external electromagnetic field.

Direct (conductive) stimulation has the disadvantage that a sequence of electrical pulses is produced transcutaneously by way of supply wires from outside the body passing through the skin of a patient. Once the bone has healed, the supply wires also have to be operatively removed. Inductive stimulation requires a considerable outlay on external apparatus for the generation of electromagnetic fields.

In practical use, both of the afore-mentioned methods for electrically stimulating bone growth have a further substantial disadvantage: because of the external devices, such as an electric pulse source for the generation of electrical stimulation pulses, supply wires and like devices, both methods can be carried out only under supervision by, for example, a medical practice or hospital. As a result, use is limited to particular times. For especially rapid healing, however, application of the methods without limitation in terms of time and on demand would be advantageous.

Finally, both the afore-mentioned methods do not take into account the state of motion of a patient. The methods are in no way adaptive and are applied to the patient without regard to the patient's absorption capacity—also referred to medically as “resonance capacity”. In that context, for example, adaptation of the intensity and frequency of electrical pulses in dependence upon the loading on the bone to be healed would be desirable for stimulation of bone growth.

The afore-mentioned disadvantages can be avoided by means of an apparatus comprising at least one piezoelectric element associated with an implant or directly with the bone, as is described in U.S. Pat. No. 6,143,035 or the corresponding ep 1 023 872 a2. That proposal avoids external apparatus and/or supply wires from an external electrical pulse source. In addition, the known proposal has the advantage that the electrical pulses generated by the piezoelectric element for the stimulation of bone growth are adaptive, that is to say the stimulation is matched to the actual state of motion and loading of a patient.

In the case of the proposal according to U.S. Pat. No. 1,143,035 it is disadvantageous that the piezoelectric element(s) is/are mounted on the outside of an implant, for example a femoral stem. The piezoelectric element(s) accordingly project(s) outwards from the implant. As a result, the implant loses the original accuracy of fit, with the consequence that there is a risk of its becoming loose. In addition, there is a risk that, as a result of external loading, the piezoelectric element(s) will become separated from the implant and therefore ineffective. When a piezoelectric element is mounted on the outside of an implant it is no longer possible to adhere to the requisite maximum tolerance of from 0.1 to 0.25 mm over the entire surface of the implant in relation to a previously reamed-out space for accommodating the implant.

The problem underlying the present invention is accordingly to provide an apparatus of the kind mentioned at the beginning wherein piezoelectric elements do not project out beyond the implant surface so that the implant can be implanted in customary manner. In addition, it should be ensured that the forces acting on the implant act directly on the piezoelectric element associated with the implant.

In accordance with the invention the problem is solved by means of the fact that at least one piezoelectric element is an integral component of the implant. The implant preferably consists at least in part of a piezoelectric ceramic.

The fact that the piezoelectric element is an integral component of the implant should ensure that the external contour of the implant remains unchanged. Consequently, the implant can be implanted in customary manner. As a result of the embedding of the piezoelectric element within the implant it is also ensured that external forces act directly and enduringly on the piezoelectric element by way of the implant. The implant always defines one electrical pole of the piezoelectric element, the second pole being defined by a contact element coming into contact only with the surrounding bone and made from an electrically conductive, especially metallic, material tolerable to humans. That contact element preferably consists likewise of implant material. It may be of strip-, disc- or button-like construction, that being dependent, in the last analysis, on the geometry of the opening of the accommodating pocket for the piezoelectric element.

In order to ensure that the implant retains its original contour in spite of the integrated piezoelectric element, the piezoelectric element is preferably so arranged inside an implant pocket which is open towards the bone that it terminates substantially flush with the surface of the implant.

Specific embodiments of implants having associated piezoelectric elements are described in claims 5 ff. They are also explained hereinbelow in greater detail with reference to the accompanying drawings, in which

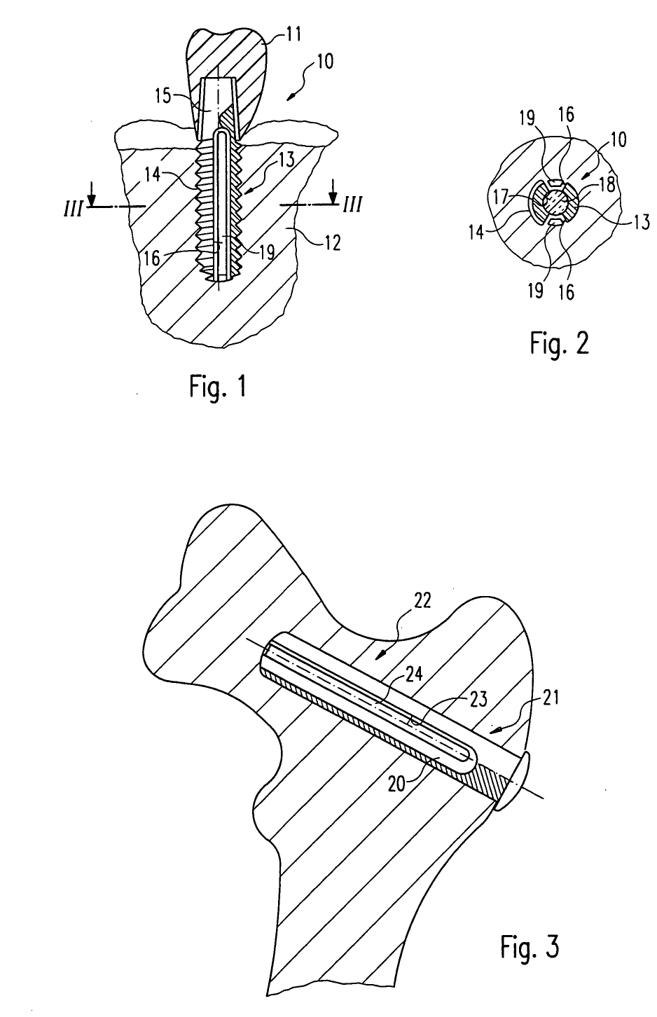

FIG. 1 shows, partly in longitudinal section and partly in side view, a tooth implant having a piezoelectric element;

FIG. 2 shows the tooth implant of FIG. 1 in cross-section along line ii-ii in FIG. 1;

FIG. 3 shows a bone-inducing femoral neck pin, partly in side view and partly in longitudinal section, showing the implantation within a femoral neck;

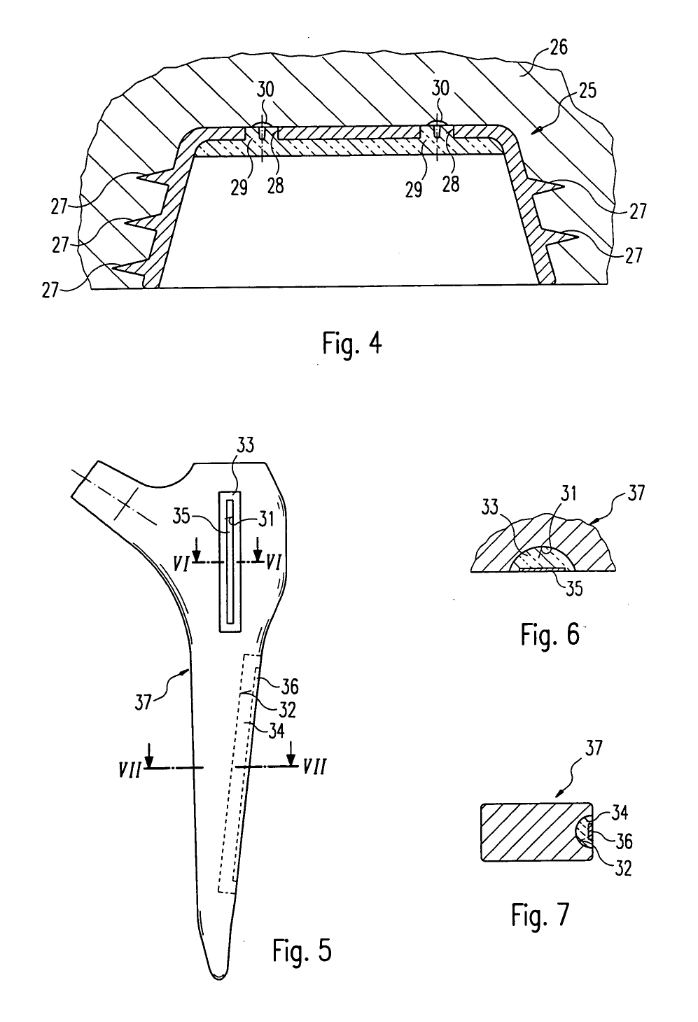

FIG. 4 shows, in section, a hip-joint socket together with a bone-inducing piezoelectric element;

FIG. 5 shows, in side view, a femoral stem having a piezoelectric element on the anterior/posterior face, on the one hand, and a further piezoelectric element laterally;

FIGS. 6 and 7 show the femoral stem of FIG. 5 in cross-section along line vi-vi and along line vii-vii in FIG. 5;

FIG. 8 shows, in section, a femoral sled showing fixation screws having a piezoelectric element;

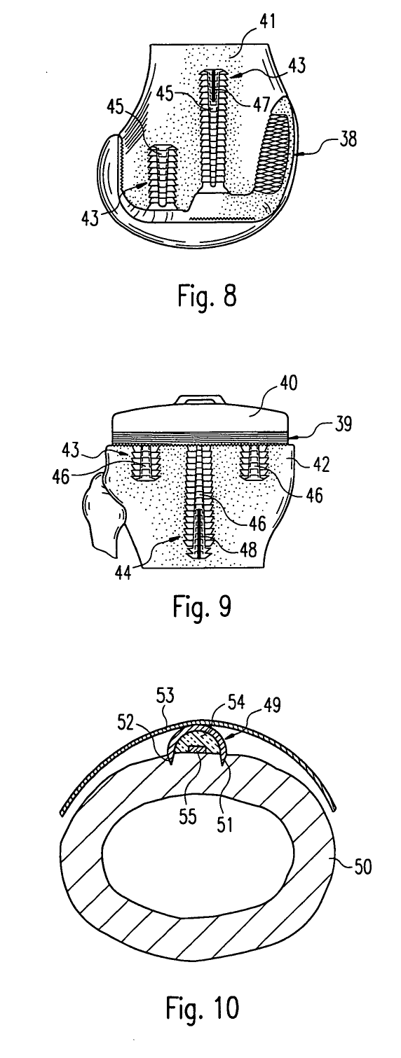

FIG. 9 shows, in section, a tibial component, the bone fixation screws of which are each provided with a piezoelectric element; and

FIG. 10 is a cross-section through a semi-circular rod element for stabilising a bone fracture, the hollow space in which, facing the bone, is filled by a piezoelectric element, in cross-section.

FIGS. 1 and 2 show, in partial longitudinal section and cross-section, a tooth implant 10 for an artificial tooth 11. The part which is anchorable in the (jaw-)bone 12 is in the form of a bone screw 13 having an external thread 14, the upper part of the bone screw 13 having a cone 15, on which the artificial tooth 11 can be placed. The threaded part of the bone screw 13 is of hollow construction and is provided with two longitudinal slots 16 arranged diametrically opposite one another. The longitudinal hollow space 17 of the threaded portion of the bone screw 13 is filled with piezoelectric ceramic, which defines the piezoelectric element according to the invention. In the region of the two longitudinal slots 16 there extend electrically conductive contact strips 19, which are made preferably from the same material as the bone screw 13, namely titanium or a titanium alloy, that is to say a material which is tolerable to humans. The contact strips 19 are in contact only with the bone 12, on the one hand, and with the piezoelectric ceramic 18, on the other hand, that is to say not with the implant screw 13. The contact strips 19 accordingly define the opposite pole to the bone screw 13. The latter preferably forms the negative pole whereas the contact strips 19 define the positive pole.

The longitudinal slots 16 are substantially filled by the contact strips 19 so that the original external contour of the bone screw 13 is virtually unchanged. The described tooth implant is especially highly effective, more specifically because of the dynamic loading of the piezoelectric element 18 during chewing. The electrical signals or pulses produced in the process bring about more rapid healing of the jaw-bone 12.

From previously obtained findings in the electrical stimulation of bone healing it is known that an effective current intensity (direct, alternating or square-wave pulse current) of about 10-100 μa is best for promoting the bone growth. The piezoelectric element is therefore preferably so constructed that, on normal loading of the bone structure, a current having an effective current intensity of about 10-100 μa is generated.

The piezoelectric element 18 preferably consists of a piezoelectric ceramic. In this context, zirconate or titanate ceramics have been found to be especially suitable for the area of surgical/orthopaedic applications, because they are tolerable to the body and can be readily integrated into the body. Other piezoelectrically active ceramics which are tolerable to the body, such as quartz ceramic, are also feasible.

FIG. 3 shows, partly in longitudinal section and partly in a side view, the implantation of a femoral neck pin 21 having a piezoelectric element 20 in the region of a femoral neck which is at risk of fracture. Reference numeral 22 denotes the femoral neck subject to the risk in question. The femoral neck pin 21, which is made from titanium or a titanium alloy, is of similar hollow construction to the bone screw 13. The hollow space is filled with piezoelectric ceramic, which defines the piezoelectric element 20. In this case too, there are provided two longitudinal slots 23 arranged diametrically opposite one another, in the region of which there are located electrically conductive contact strips 24 corresponding to the contact strips 19 according to FIGS. 1 and 2, more specifically in such a manner that they are in contact with the ceramic 20, on the one hand, and with the surrounding bone, on the other hand. The contact strips 24 therefore form the opposite pole to the pin 21. It should be mentioned at this point that the bone structure has a crystalline structure and reacts on mechanical loading with piezoelectric pulses. In the converse case, the bone reacts with a mechanical moment, which again results in bone formation. That reciprocal action of mechanical moments and piezoelectric pulses is utilised in accordance with the invention.

The femoral neck pin shown in FIG. 3 is used in this instance for prophylaxis but can be used equally well for healing a femoral head fracture.

In very similar manner, bone or pedicle screws can be introduced at other sites in the bone for the purpose of prophylaxis. Bone or pedicle screws of such a kind have a threaded part corresponding to that of the bone screw 13 in FIGS. 1 and 2.

FIG. 2 shows a hip-joint socket or hip socket 25, which is screwed into the hip bone 26. Reference numeral 27 in FIG. 4 denotes the corresponding screw thread. In the case of old and very old patients, the bone recedes in the region of a cementless hip socket implant, such as the hip socket 25 shown here. The hip socket is then held in position only by thin bone trabeculae. In order to prevent that problem, the hip socket 25 shown is provided with openings 28 in its bottom, which are each filled with piezoelectric ceramic 29. The piezoelectric ceramic also extends over the entire inside of the bottom of the socket and is subjected to pressure by the inlay (not shown in FIG. 4). On the outside of the socket, the piezoelectric ceramic 29 is in contact with the bone 26 by way of push-button-like contact elements 30. The push-button-like contact elements 30 are in contact with the piezoelectric ceramic 29, on the one hand, and with the surrounding bone 26, on the other hand; otherwise, they are isolated from the socket 25. The contact elements 30 accordingly form the electrically opposite pole to the socket 25. In the cementless hip socket implant shown, bone growth is promoted, on the one hand, by the tips of the thread 27 and, on the other hand, by the piezoelectric system shown. Especially as a result of the latter, bone formation takes place in the direction of the implant, which becomes increasingly stabilised in the course of time. As a result of the measures described, therefore, exactly the opposite effect occurs to that which would normally be expected, namely bone formation instead of bone loss.

FIGS. 5 to 6 show a femoral stem having a pocket 31 and 32 formed in an anterior and a lateral position for accommodating a piezoelectric ceramic or piezoelectric element 33 and 34, respectively. The accommodating pockets 31 and 32 are each in the form of longitudinal grooves and each is of approximately semi-circular cross-section. Contact strips 35 and 36 are embedded in the piezoelectric ceramic 33 and 34, respectively, on the side facing the bone, more specifically in such a manner that the piezoelectric ceramic including the contact strip terminates flush with the external surface of the implant, in this case the femoral stem 37.

The femoral stem 37 can also be provided with elements 33, 34 corresponding to piezoelectric elements on the posterior and/or the medial face, that being dependent, in the final analysis, on the structure of the patient's bone. In this instance too, the contact strips 35, 36 again form the positive electrical pole of the piezoelectric element 33 and 34, respectively, whereas the implant itself, namely the femoral stem 37, defines the negative pole.

The examples described also show very clearly that no wires are installed for the transmission of current pulses. The implants are intended to have substantially their original shape so that they can be implanted in customary manner.

FIGS. 8 and 9 show, in diagrammatic longitudinal section, a femoral sled 38 on the one hand and a tibial plateau 39 on the other hand, a bearing body 40 made of polyethylene or like plastics material which is tolerable to humans being mounted fixedly or displaceably (translation and/or rotation) on the latter. Both the femoral sled and also the tibial plateau are fixed to the femur 41 and the tibia 42, respectively, by means of bone screws 43 and 44. The bone screws 43, 44 have a threaded part, which corresponds to that of the bone screw 13 according to FIG. 1. In the case of the bone screws 43, 44 too, a longitudinal hollow space is provided, which is filled with a piezoelectric ceramic, forming in each case a piezoelectric element 45, 46. In the region of the longitudinal slots there are again provided contact strips 47, 48, which are in contact with the piezoelectric ceramic on the one hand and with the surrounding bone on the other hand.

The piezoelectric element 45 and 46 is, in each case, somewhat conical, more specifically widening out conically towards the end of the screw so that the threaded part of the bone screws 43, 44 is correspondingly expanded outwards in a radial direction, thereby achieving a better hold in cancellous bone.

FIG. 10 shows, in cross-section, an elongate stabilising element in accordance with de 4 102 462 a1, more specifically in association with a bone 50. Reference numeral 49 denotes the stabilising element, which is in the form of an elongate half-tube. It has only linear contact with the bone surface, that linear contact being interrupted by tips 51, 52 spaced longitudinally apart from one another, which penetrate into the bone. The stabilising element shown is held in position by means of a holding band 53 wrapped around the bone and the stabilising element 49. FIG. 10 shows only part of the holding band 53. Above all, the closing element for the two free ends of the holding band is not shown. To that extent, however, it represents known prior art, also originated by the inventor.

The hollow space between the stabilising element 49 and the surface of the bone is filled with a piezoelectric ceramic. On the side facing the surface of the bone, an electrically conductive contact strip 55 is embedded in the ceramic 54. The contact strip is isolated from the stabilising element 54 by the ceramic as in the previously described embodiments and defines the opposite pole to the stabilising element 49, which is made from titanium or a titanium alloy. In this instance too, the piezoelectric element 54 is an integral part of the stabilising element 49.

When more than one piezoelectric element is provided, at least two piezoelectric elements can be electrically connected in series in order to obtain a higher electrical voltage. Alternatively, the piezoelectric elements can also be electrically connected in parallel, as a result of which a higher current intensity can be obtained, it being fundamental that the effective current intensity of 10-100 μa is achieved. The piezoelectric elements should then be connected in the corresponding manner.

As already mentioned, the piezoelectric elements can be associated with a very great diversity of implants including, for example, an artificial patella. In that respect there are no limits.

Loading of the piezoelectric element is effected by way of the implant, on the one hand, and the bone, on the other hand, it also being possible for pressure to be exerted by the musculature.

All features described in the application documents are claimed to be of inventive significance insofar as they are, on their own or in combination, novel with respect to the prior art.

List of Reference Numerals

10 tooth implant

11 artificial tooth

12 (jaw-)bone

13 bone screw

14 external thread

15 insertion cone

16 longitudinal slot

17 longitudinal hollow space

18 piezoelectric element (ceramic)

19 contact strip

20 piezoelectric element (ceramic)

21 femoral neck pin

22 femoral neck

23 longitudinal slot

24 contact strip

25 hip socket

26 hip bone

27 screw thread

28 opening in bottom

29 piezoelectric element (ceramic)

30 contact element

31 pocket

32 pocket

33 piezoelectric element (ceramic)

34 piezoelectric element (ceramic)

35 contact strip

36 contact strip

37 femoral stem

38 femoral sled

39 tibial plateau

40 bearing body

41 femur

42 tibia

43 bone screw

44 bone screw

45 piezoelectric element (ceramic)

46 piezoelectric element (ceramic)

47 contact strip

48 contact strip

49 stabilising element

50 bone

51 tip

52 tip

53 holding band

54 piezoelectric element (ceramic)

55 contact strip

1-11. (canceled)

12. An apparatus for promoting growth selected from the group consisting of bone growth, osteosynthesis of bone fragments, fixation of bone fractures, and osteosynthesis of bone fragments with fixation of bone fractures, said apparatus comprising:
at least one implant;
at least one piezoelectric element associated with said implant, said piezoelectric element under the action of forces, generates electrical pulses which serve as a stimulant for bone growth, said at least one piezoelectric element forming an integral component of said at least one implant; and
at least one contact element operative to come into contact only with surrounding bone and said piezoelectric element, said at least one contact element being made from electrically conductive material tolerable to humans; and wherein
said implant defining one pole and said contact element defining the other pole of said piezoelectric element; and
said piezoelectric element being arranged within said implant or within an implant pocket open towards the bone.

13. The apparatus of claim 12, wherein said piezoelectric element terminates substantially flush with the surface of said implant.

14. The apparatus of claim 12, wherein said implant is in the form of a dowel defining a central hollow space, said piezoelectric element being located in said central hollow space of said implant.

15. The apparatus of claim 12, wherein said implant is selected from the group consisting of a pin-like holder for an artificial tooth, a bone or pedicle screw, a bone fixation pin, and a bone fixation element.

16. The apparatus of claim 12, wherein said implant is a hip-joint socket defining at least one opening in the bottom thereof, said piezoelectric element being arranged to be located in said opening in the bottom of said hip-joint socket.

17. The apparatus of claim 16, wherein said piezoelectric element is arranged in and fills said opening in the bottom of said hip-joint socket and is integrally connected to a piezoelectric layer extending over at least part of the inside of the bottom of said hip-joint socket.

18. The apparatus of claim 12, wherein said piezoelectric element is so constructed that, on normal loading of the bone structure, a current having an effective current intensity of about 10-100 μA is generated.

19. The apparatus of claim 12, wherein said piezoelectric element is made from a material selected from the group consisting of a piezoelectric ceramic, a zirconate ceramic and a titanate ceramic.

20. The apparatus of claim 12, wherein at least two of said piezoelectric elements are provided and have an electrical connection selected from the group consisting of series electrical connection, and parallel electrical connection.

21. The apparatus of claim 12, wherein said at least one implant is made of metallic material.

22. The apparatus of claim 12, wherein said implant defines a negative pole and said contact element defines a positive pole.
